(19) United States
(12) Patent Application Publication
(10) Pub. No.: US 2020/0022727 A1
(43) Pub. Date: Jan. 23, 2020

(54) SYSTEMS, DEVICES, AND METHODS FOR TREATING AND MONITORING A PREGNANT PATIENT HAVING A PREMATURELY OPEN CERVIX

(71) Applicant: PARTUS LLC, Laguna Niguel, CA (US)

(72) Inventors: William GILBERT, Laguna Niguel, CA (US); Thomas GARDNER, Laguna Niguel, CA (US); Gonzalo COVARRUBIAS, Laguna Niguel, CA (US); Souhail TOUBIA, Laguna Niguel, CA (US)

(21) Appl. No.: 16/483,008

(22) PCT Filed: Jan. 31, 2018
(86) PCT No.: PCT/US18/16282

§ 371 Date: Aug. 1, 2019

Related U.S. Application Data
(60) Provisional application No. 62/453,445, filed on Feb. 1, 2017.

(51) Int. Cl. A61B 17/42; A61B 17/122

(52) U.S. Cl. CPC A61B 17/42; A61B 2017/00022; A61B 2017/4225; A61B 17/122

(57) ABSTRACT

Described herein are systems, devices, and methods for treating and/or monitoring a patient with a prematurely dilated cervix during pregnancy or a patient with a history of a prematurely dilated cervix in a previous pregnancy. The systems, devices, and methods described herein further provide monitoring of the status of a cervix by, for example, sensing a change associated with the cervix of a patient. The systems, devices, and methods described herein are configured to either partially or completely encircle the cervix of a patient and constrict the encircled cervix. In some embodiments, the systems, devices, and methods described herein comprise a first and a second coupler that reversibly couple together so that these embodiments are configured to be manually placed (i.e. the couplers are coupled together) and then either removed or adjusted by a healthcare provider (i.e. the couplers are decoupled from each other).

### CROSS-REFERENCE

This application claims the benefit of U.S. Provisional Application No. 62/453,445, filed Feb. 1, 2017, which application is incorporated herein by reference.

### BACKGROUND

Cervical incompetence or insufficiency describes a condition in which the cervix, which is the opening of the uterus, shortens and opens prematurely during a pregnancy. This condition is associated with late term miscarriage as well as pre-term birth.

The traditional treatment is known as cerclage or cervical stitch. It is the placement and tightening of a suture tie around the prematurely dilated cervix, thus closing it.

### SUMMARY

Described herein are systems, devices, and methods for treating and/or monitoring a patient with a prematurely dilated cervix during pregnancy or a patient with a history of a prematurely dilated cervix in a previous pregnancy. The systems, devices, and methods described herein further provide monitoring of the status of a cervix and/or a fetus by, for example, sensing a change associated with the cervix of a patient and/or a fetus.

Traditional cerclage places a suture through the cervix thus closing the prematurely open cervix, and this traditional approach, is typically performed in the operating room while the patient is under anesthesia. Both the traditional procedure itself as well as the way in which it is performed are associated with risks to the patient as well as increased burden on the patient and the healthcare provider.

The risks and burdens associated with the traditional treatment of a prematurely dilated cervix are overcome by the systems, devices, and methods described herein.

An example of a complication associated with traditional cerclage is uterine laceration with bleeding (which is also further associated with infection). The uterus (of which the cervix is the opening) is increasingly vascular and friable during pregnancy, and thus placement of a suture needle through the cervix (in order to place the suture) in traditional cerclage along with the grasping of the uterus with surgical instruments is associated with cervical laceration and bleeding. Cervical laceration caused during the traditional cerclage procedure may be associated with serious blood loss as bleeding from the highly vascularized pregnant uterus can be difficult to control. In addition, laceration and bleeding create wounds that are susceptible to the complication of wound infection. In contrast, in some embodiments of the systems, devices, and methods described herein, a cervix that prematurely opens during pregnancy is treated by the application of a releasable clamp that is placed around the cervix. In these embodiments, the clamp is sutureless and may be placed manually thus avoiding both passage of a sharp suture needle through the cervix as well as, in some embodiments, eliminating the need to grab the cervix of the patient with surgical instruments.

Traditional cerclage is also associated with a number of anesthesia related complications as the traditional cerclage procedure is typically performed under anesthesia. Complications associated with anesthesia include, for example, hypotension, asphyxia, pulmonary embolus, and cardiac arrest. In some embodiments of the systems, devices, and methods described herein, a clamp for encircling (at least partially) and closing a prematurely opened cervix in a pregnant patient is configured for placement in the setting of a clinic or doctor's office and does not require use of anesthesia thus obviating the number of complications associated with anesthesia. In addition to the known risks and potential complications of traditional cerclage, the required use of the operating room is often inconvenient for patients and physicians and adds an extra cost onto the procedure, which can be avoided by the embodiments of the systems, devices, and methods described herein configured for use in the clinic or doctor's office settings.

The systems, devices, and methods described herein are configured to either partially or completely encircle the cervix of a patient and constrict the encircled cervix. In some embodiments, the systems, devices, and methods described herein comprise a first and a second coupler that reversibly couple together so that these embodiments are configured to be manually placed (i.e. the couplers are coupled together) and then either removed or adjusted by a healthcare provider (i.e. the couplers are decoupled from each other by the healthcare provider).

Additionally, some embodiments of the systems, devices, and methods described herein comprise one or more sensors configured to sense a physiologic change in the patient or the fetus. In these embodiments, a sensor used may be configured to, for example, sense a physiologic change in the uterus, the fetus, or the mother.

For example, the sensor used in some embodiments of the systems, devices, and methods described herein may be configured to sense a pressure change associated with the cervix. That is, such a sensor may be configured to sense a change in radial pressure exerted by the cervix against the sensor as the cervix expands and contracts. A sensor that senses a change in radial pressure exerted by a cervix is beneficial in that, for example, it provides information used in determining if the cervix is dilating further which may indicate that pregnancy is progressing towards labor.

For example, the sensor used in some embodiments of the systems, devices, and methods described herein (said systems, devices, and methods for placement so that they encircle the cervix at least partially) senses a heart rate of a fetus as transmitted through the uterus of the mother. A sensor that, for example, continuously senses the heart rate of the fetus provides information relating to the health and viability of the fetus.

For example, a sensor used with the systems, devices, and methods described herein senses the blood flow to the cervix by, for example, sensing the oxygen saturation of the cervix tissue. A sensor that, for example, senses blood flow to the walls of the uterus (e.g. at the cervix) provides data used to determine whether a device placed to close a prematurely dilated cervix is causing a decrease in blood flow by constraining blood flow. Thus, such a sensor used in these embodiments, provides information used to determine whether a device that is placed to treat a prematurely dilated cervix (or a closed cervix in a patient with a history of prematurely dilated cervix in a previous pregnancy) is constraining blood flow to the cervix and thus needs to be either loosened, repositioned, or otherwise adjusted.

In some embodiments, one or more sensors as described herein are components of a system in which physiologic data sensed by one or more sensors in proximity to or in contact with the cervix transmit sensed physiologic data to a receiver that is external to the patient. In these embodiments, a receiver may be coupled to processor of a computing device such as, for example, a mobile computing device. Exemplary mobile computing devices include smartphones, smart watches, and laptops. Likewise, a desktop computer may be coupled to the receiver as well. A computing device that receives data from a transmitter coupled to one or more sensors as described herein may further comprise one or more software modules configured to cause the processor of said computing device to process the received data in order to make a determination based on said physiologic data. For example, one or more types of physiologic data received from one or more types of sensors may be used by the computing device to determine a physiologic state of either the mother of the fetus. In some embodiments, software operating on the computing device provides a user interface, which may be used, for example, to display received physiologic data or physiologic conditions of the fetus and/or mother.

In some embodiments of the systems described herein, a system as described includes a remote host configured to either receive sensed physiologic data from a local computing device or receive physiologic data directly from one or more sensors that are either in proximity to or in contact with the cervix (and therefore the uterus) of the patient. In some embodiments, a remote host is positioned at a remote monitoring center. In some embodiments, a remote host is configured to communicate with one or more mobile computing devices.

Described herein is a device for preventing a pre-mature opening of a cervix of a patient, comprising: a first arm having a first proximal end and a first distal end; a second arm having a second proximal end and a second distal end; a hinge that rotatably couples the first arm at the first proximal end with the second arm at the second proximal end, the first arm and the second arm being positioned so that the first arm and the second arm surround the cervix of the patient when the first arm and the second arm are rotated towards each other about the hinge; a first coupler positioned at the first distal end and a second coupler positioned at the second distal end, wherein the first coupler and the second coupler are configured to couple with each other such that the first arm and the second arm are secured against the cervix of the patient; and a sensor configured to sense a change associated with the cervix of the patient. In some embodiments, the first arm and the second arm each have one or more teeth for securing the first arm and the second arm to the cervix of the patient. In some embodiments, the first arm and the second arm each comprises a curved segment configured to conform to the shape of the cervix of the patient. In some embodiments, the first coupler and the second coupler are configured to reversibly couple with each other. In some embodiments, the sensor comprises a pressure sensor configured to sense a pressure exerted by the cervix of the patient on the device. In some embodiments, the device comprises a transmitter configured to transmit the pressure exerted by the cervix of the patient on the device that is sensed by the sensor. In some embodiments, the first coupler comprises an opening having one or more first protrusions and first recesses and the second coupler comprises a flat segment configured to fit the opening and having one or more second protrusions and second recesses, and wherein the first coupler and second coupler couple in a ratcheting fashion wherein the first protrusions couple with the second recesses and the first recesses couple with the second protrusions.

Also described herein is a method for preventing a pre-mature opening of a cervix of a patient comprising: securing a clamp, having a sensor, around the cervix of the patient without use of a suture, thereby closing an opening of the cervix of the patient; and monitoring, using the sensor, a change associated with the cervix of the patient. In some embodiments, the clamp comprises a first arm having a first proximal end and a first distal end, a second arm having a second proximal end and a second distal end, a hinge that rotatably couples the first arm at the first proximal end with the second arm at the second proximal end, the first arm and the second arm being positioned so that the first arm and the second arm surround the cervix of the patient when the first arm and the second arm are rotated towards each other about the hinge, a first coupler positioned at the first distal end, and a second coupler positioned at the second distal end, wherein the first coupler and the second coupler are configured to couple with each other such that the first arm and the second arm are secured against the cervix of the patient. In some embodiments, first arm and the second arm each have one or more teeth for securing the first arm and the second arm to the cervix of the patient. In some embodiments, the first arm and the second arm each comprises a curved segment configured to conform to the shape of the cervix of the patient. In some embodiments, the first coupler and the second coupler are configured to reversibly couple with each other. In some embodiments, the sensor comprises a pressure sensor configured to sense a pressure exerted by the cervix of the patient on the clamp. In some embodiments, the clamp comprises a transmitter configured to transmit the pressure exerted by the cervix of the patient on the device that is sensed by the sensor. In some embodiments, the first coupler comprises an opening having one or more first protrusions and first recesses and the second coupler comprising a flat segment configured to fit the opening and having one or more second protrusions and second recesses, and wherein the first coupler and second coupler couple in a ratcheting fashion wherein the first protrusions couple with the second recesses and the first recesses couple with the second protrusions.

Also described herein is a system for preventing a pre-mature opening of a cervix of a patient comprising: a clamp comprising a first arm having a first proximal end and a first distal end; a second arm having a second proximal end and a second distal end; a hinge that rotatably couples the first arm at the first proximal end with the second arm at the second proximal end, the first arm and the second arm being positioned so that the first arm and the second arm surround the cervix of the patient when the first arm and the second arm are rotated towards each other about the hinge; a first coupler positioned at the first distal end and a second coupler positioned at the second distal end, wherein the first coupler and the second coupler are configured to couple with each other such that the first arm and the second arm are secured against the cervix of the patient; a sensor configured to sense data associated with the cervix of the patient; a transmitter configured to receive the data from the sensor and wirelessly transmit the data; a host comprising: a receiver configured to receive the data that is transmitted by the transmitter; a processor; and a non-transitory computer-readable storage media encoded with a computer program including instructions executable by the processor to cause the processor: determine if the data is within pre-set limits; and generate an alert when the data is outside of the pre-set limits.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary embodiment of a device for closing a prematurely open cervix during pregnancy.

FIG. 2 shows an exemplary hinge-less embodiment of a device for closing a prematurely open cervix during pregnancy.

FIG. 3 shows an exemplary embodiment of a device for closing a prematurely open cervix during pregnancy that includes a skirt.

FIG. 4 shows an exemplary embodiment of a device for closing a prematurely open cervix during pregnancy that includes a skirt and does not include a clamp.

FIG. 5 shows an exemplary embodiment of a system for treating and monitoring a pregnant patient with a prematurely open cervix during pregnancy.

FIG. 6 shows the steps of an exemplary method for treating and monitoring a pregnant patient with a prematurely open cervix.

### DETAILED DESCRIPTION

Described herein are systems, devices, and methods for treating and/or monitoring a patient with a prematurely dilated cervix during pregnancy or a patient with a history of a prematurely dilated cervix in a previous pregnancy (said patient with a history of a prematurely dilated cervix having either an open or closed cervix in the current pregnancy). The systems, devices, and methods described herein further provide monitoring of the status of a cervix by, for example, sensing a change associated with the cervix of a patient.

Devices for Treating and Monitoring a Pregnant Patient with a Prematurely Open Cervix

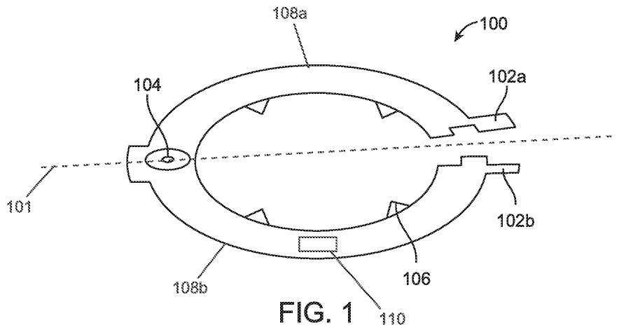

FIG. 1 shows an exemplary embodiment of a device for closing a prematurely open cervix during pregnancy. In some embodiments described herein, a device for closing a prematurely open cervix comprises a clamp 100 configured to be manually positioned to at least partially encircle the cervix so that closure of the clamp closes the cervix of the patient. That is, the clamp 100 is placed so that it encircles at least a portion of the cervix of the patient so that a pressure applied by the closure of the placed clamp 100 against the walls of the cervix closes (at least partially) the cervix of the patient.

In some embodiments, manual delivery of the clamp 100 may be achieved without further use of a delivery device (aside from a speculum for aiding in visualization of the cervix of the patient).

In some embodiments, a delivery device is configured to position the clamp 100 so that it encircles at least a portion of the cervix, and in some embodiments, a delivery device assists in the closing of the clamp 100 so that it encircles at least a portion of the cervix.

In the embodiment shown in FIG. 1, a clamp 100 comprises a first arm 108a and a second arm 108b. In some embodiments of clamp 100, first arm 108a and second arm 108b comprise a rigid material so that their shape is essentially fixed. In some embodiments of clamp 100, first arm 108a and second arm 108b comprise a malleable material so that they are configured to be manually shaped against the contours of the cervix of the patient. In some embodiments of the clamp 100, the clamp 100 comprises an ellipse like shape or circular shape as shown in FIG. 1. In the ellipse shaped and circular shaped embodiments of the clamp 100, the first arm 108a and the second arm 108b each comprise a curved segment so that when they are coupled with each other the curved segments of each of the first arm 108a and the second arm 108b form an ellipse or circular shape. However, it should be understood that clamp 100 may comprise any number of other shapes including and non-limited to triangular shapes, square shapes, and rectangular shapes, and that for each possible shape, the shape of the clamp 100 is formed by the shapes of the segments of arms 108a and 108b.

As shown in FIG. 1, in some embodiments of clamp 100, the first arm 108a and the second arm 108b are rotatably coupled, at least at one point, about a hinge 104. Rotation of the first arm 108a and the second arm 108b about the hinge 104 in a direction towards a midline 101 of the clamp 100 moves the first arm 108a and the second arm 108b towards each other thus closing the device, and rotation of the first arm 108a and the second arm 108b about the hinge 104 away from a midline 101 of the clamp 100 moves the first arm 108a and the second arm 108b away from each other thus opening the clamp 100.

In some embodiments, each of the first arm 108a and the second arm 108b are configured to rotate independently about the hinge 104, and in some embodiments, hinge 104 is configured to cause either of the first arm 108a or the second arm 108b to automatically rotate when the other arm rotates. That is, in some embodiments, one of the arms (i.e. either 108a or 108b) is rotated automatically by hinge 104 in response to the manual rotation of the other arm about the hinge 104. For example, in the embodiments of clamp 100 that include the automatically rotating arm feature, when the first arm 108a is rotated by a user towards a midline 101 of the clamp 100, the second arm 108b is automatically rotated by the hinge 104 towards the midline 101 of the clamp 100 and travels a distance equal to the distance traveled by the manually rotated first arm 108a.

The automatic rotation of an arm about the hinge 104 is beneficial in that it, for example, provides a healthcare provider with the ability to close the clamp 100 around the cervix of a patient manually without having to press both arms 108a, 108b of the clamp 100 towards the midline 101 of the clamp 100, because pressing one of the arms against the cervix of the patient (i.e. towards the midline of the device) causes the other arm to automatically press against the cervix of the patient. This feature is particularly beneficial in, for example, instances when there is not enough room within the pelvis for the physician to be able to contact both arms of the clamp 100 at once in order to encircle the open cervix of the patient with the clamp 100. In these automatically closing embodiments of clamp 100, a healthcare provider positions the clamp 100 to encircle the cervix by, for example, pressing first arm 108a against the cervix of the patient, thus rotating first arm 108a about the hinge 104, and in response, the hinge 104 causes second arm 108b to rotate an equal distance about the hinge 104 also moving second arm 108b against the cervix of the patient.

In embodiments of the clamp 100 where the first arm 108a and the second arm 108b rotate independently about hinge 104, a healthcare provider closes the clamp 100 about the cervix by pressing the first arm 108a and the second arm 108b against the cervix of the patient.

As shown in FIG. 1, the clamp 100 further comprises a first coupler 102a positioned at an end of first arm 108a and a second coupler 102b positioned at an end of second arm 108b. In some embodiments of clamp 100, the first coupler 102a and the second coupler 102b are configured to reversibly couple to each other so that they may be manually coupled and decoupled by a user allowing for repositioning, adjustment, and remove of clamp 100 manually by healthcare provider.

In some embodiments of clamp 100, couplers 102a and 102b are configured and positioned to interlock along different points along first arm 108a and second arm 108b so that the diameter of the clamp 100 is adjustable based on the location along the first arm 108a and the second arm 108b at which the first coupler 102a and the second coupler 102b couple. In some embodiments, a plurality of couplers are positioned at different locations along the first arm 108a and the second arm 108b to provide multiple points of coupling along each of the arms 108a, 108b.

Couplers 102a and 102b are configured to interlock. Non-limiting examples of interlocking mechanisms include snap-fit mechanisms, ratcheting (i.e. zip-tie) mechanisms, hooking mechanisms, and buttoning mechanisms. It should be understood that numerous mechanisms are suitable for use with coupler 100.

In the embodiment of the clamp 100 shown in FIG. 1, first coupler 102a is configured to receive a protrusion in second coupler 102b so that the first coupler 102a and the second coupler 102b couple together in a snap-fit fashion and thereby couple the first arm 108a to the second arm 108b.

In some embodiments of the clamp 100, couplers 102a and 102b are configured to couple in a ratcheting fashion. In embodiments having ratcheting couplers, the first coupler 102a comprises an opening having one or more protrusions and one or more recesses, wherein each of the one or more protrusions is adjacent to each of the one or more recesses forming a protrusion-recess-protrusion-recess pattern. The second coupler 102b comprises a flat segment configured to fit the opening of the first coupler 102a and having one or more protrusions and one or more recesses, wherein each of the one or more protrusions is adjacent to each of the one or more recesses forming a protrusion-recess-protrusion-recess pattern. The first coupler 102a is configured to receive the second coupler 102b within the opening of the first coupler 102a so that the protrusions of the first coupler 102a fit tightly into the recesses of the second coupler 102b and the protrusions of the second coupler are fit tightly into the recesses of the first coupler 102a.

In some embodiments of the clamp 100, couplers 102a and 102b are configured to couple in a hooking fashion. In some embodiments, one of the couplers 102a and 102b comprises an opening and the other coupler (either 102a or 102b) comprises a hook or pin configured to fit the opening. In some embodiments, both couplers 102a and 102b comprise openings fitted to receive a hook, pin, or tie that is passed through both openings thus coupling the both couplers 102a and 102b together.

In some embodiments of the clamp 100, couplers 102a and 102b are configured to couple in a buttoning fashion. In some embodiments, one of the couplers 102a and 102b comprises a button shape and the other coupler (either 102a or 102b) comprises an opening that is configured to securely fit over the button thus coupling both couplers 102a and 102b.

In some embodiments of the clamp 100, the clamp 100 includes one or more fixing elements 106 configured to securely fix the clamp 100 to the cervix of the patient. In some embodiments of clamp 100, one or more fixing elements 106 comprise one or more teeth as shown in FIG. 1. Embodiments of clamp 100 that include one or more fixing elements 106 are beneficial in that they, for example, secure the clamp 100 to the cervix of the patient in a manner that requires less pressure be applied by the first and second arms 108a, 108b against the walls of the cervix of the patient then would be applied if there were no fixing elements 106 and the first and second arms 108a, 108b alone secured the clamp 100 to the cervix of the patient. Having less pressure applied by the first and second arms 108a, 108b is beneficial in patients at risk for ischemia. Embodiments of clamp 100 that do not include any fixing elements 106 are beneficial in that they have a lower risk of causing bleeding from the cervix of the patient than the embodiments of clamp 100 that include the penetrating fixing elements 106, because these embodiments are configured to not penetrate the cervix of the patient.

In some embodiments of clamp 100, clamp 100 includes one or more sensors 110. The one or more sensors 110 are positioned so that they are able to sense physiologic parameters when the clamp 100 is positioned to encircle the cervix of the patient. In some embodiments of clamp 100, clamp 100 comprises a pressure sensor such as, for example, a tactile pressure sensor that is configured to sense a radial pressure exerted by the cervix of the patient against the sensor. A radial pressure exerted by the cervix of the patient that is sensed by the pressure sensor may, for example, indicate a contraction of the uterus or may, for example, indicate continued opening of the cervix of the patient. In some embodiments of clamp 100, clamp 100 includes a sensor configured to sense blood flow to the cervix of the patient such as, for example, a pulse oxygen sensor. A change in blood flow to the cervix may, for example, indicate that the clamp 100 is too tight and is constricting blood flow. In some embodiments of clamp 100, clamp 100 comprises a fetal heart rate sensor configured to sense a heart rate of a fetus and thus provides information regarding the health of the fetus. It should be understood that numerous other sensor types are suitable for use with the systems, devices, and methods described herein including and not limited to blood pressure sensors, glucose sensors, and heart rate sensors (sensing maternal heart rate). It should be also understood that some embodiments of clamp 100 include one sensor and some embodiments of clamp 100 include a plurality of sensors. In embodiments that include one sensor, the one sensor may sense either a single or multiple parameters, and in embodiments that include a plurality of sensors each of the sensors may sense either a single or multiple parameters. One or more sensors may positioned to be in contact with the cervix of the patient when the clamp 100 is positioned to encircle the cervix of the patient or the one or more sensors may be positioned in a different location within the pelvis (i.e. not in contact with the cervix of the patient) when the clamp 100 is positioned to encircle the cervix of the patient.

In some embodiments, a clamp 100 includes a transmitter that is functionally coupled with one or more sensors of a clamp 100. The transmitter is configured to transmit a wireless signal to a receiver that is positioned outside of the body of the patient.

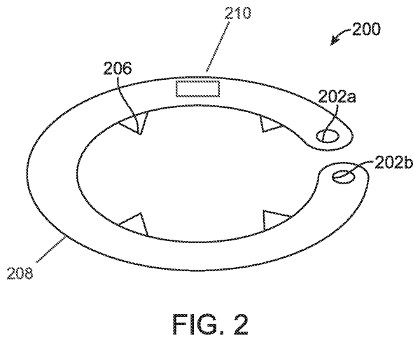

FIG. 2 shows an exemplary hinge-less embodiment of a device for closing a prematurely open cervix during pregnancy. A clamp 200 is configured to be manually positioned to at least partially encircle the cervix so that closure of the clamp closes the cervix of the patient. That is, the clamp 200 is placed so that it encircles at least a portion of the cervix of the patient so that a pressure applied by the closure of the placed clamp 200 against the walls of the cervix of the patient closes (at least partially) the cervix of the patient.

In some embodiments, manual delivery of the clamp 200 is achieved without further use of a delivery device (aside from a speculum for aiding in visualization of the cervix of the patient). In some embodiments, a delivery device is configured to position the clamp 200 so that it encircles at least a portion of the cervix, and in some embodiments, a delivery device assists in the closing of the clamp 100 so that it encircles at least a portion of the cervix.

In the embodiment shown in FIG. 2, a clamp 200 comprises a single curved elongate body 208. In some embodiments of clamp 200, the curved elongate body 208 comprises a rigid material so that the shape of the elongate body 208 is essentially fixed. In some embodiments of clamp 200, the elongate body 208 comprises a malleable material so that they are configured to be manually shaped against the contours of the cervix of the patient. In some embodiments of the clamp 200, the clamp 200 comprises an ellipse like shape or circular shape as shown in FIG. 2. In the ellipse shaped and circular shaped embodiments of the clamp 200, the elongate body 208 comprises a curved shape. However, it should be understood that clamp 200 may comprise any number of other shapes including and non-limited to triangular shapes, square shapes, and rectangular shapes, and that for each possible shape, the shape of the clamp 200 is formed by the shape of the elongate body 208.

As shown in FIG. 2, the clamp 200 further comprises a first coupler 202a positioned at a first end of the elongate body 208 and a second coupler 202b positioned at a second end of the elongate body 208. In some embodiments of clamp 200, the first coupler 202a and the second coupler 202b are configured to reversibly couple to each other so that they may be manually coupled and decoupled by a user allowing for repositioning, adjustment, and remove of clamp 200 manually by healthcare provider.

In some embodiments of clamp 200, couplers 202a and 202b are configured and positioned to interlock along different points along first arm 208a and second arm 208b so that the diameter of the clamp 200 is adjustable based on the location along the first arm 208a and the second arm 108b at which the first coupler 102a and the second coupler 202b couple. In some embodiments, a plurality of couplers are positioned at different locations along the first arm 208a and the second arm 208b to provide multiple points of coupling along each of the arms 208a, 208b.

Couplers 202a and 202b are configured to interlock. Non-limiting examples of interlocking mechanisms include snap-fit mechanisms, ratcheting (i.e. zip-tie) mechanisms, hooking mechanisms, and buttoning mechanisms. It should be understood that numerous mechanisms are suitable for use with coupler 200.

In some embodiments of the clamp 200, first coupler 202a is configured to receive a protrusion in second coupler 102b so that the first coupler 202a and the second coupler 102b couple together in a snap-fit fashion and thereby couple the first and second couplers 202a and 202b.

In some embodiments of the clamp 200, couplers 202a and 202b are configured to couple in a ratcheting fashion. In embodiments having ratcheting couplers, the first coupler 202a comprises an opening having one or more protrusions and one or more recesses, wherein each of the one or more protrusions is adjacent to each of the one or more recesses forming a protrusion-recess-protrusion-recess pattern. The second coupler 202b comprises a flat segment configured to fit the opening of the first coupler 202a and having one or more protrusions and one or more recesses, wherein each of the one or more protrusions is adjacent to each of the one or more recesses forming a protrusion-recess-protrusion-recess pattern. The first coupler 202a is configured to receive the second coupler 202b within the opening of the first coupler 202a so that the protrusions of the first coupler 202a fit tightly into the recesses of the second coupler 202b and the protrusions of the second coupler are fit tightly into the recesses of the first coupler 202a.

In some embodiments of the clamp 200, couplers 202a and 202b are configured to couple in a hooking fashion. In some embodiments, one of the couplers 202a and 202b comprises an opening and the other coupler (either 202a or 202b) comprises a hook or pin configured to fit the opening. In some embodiments, both couplers 202a and 202b comprise openings fitted to receive a hook, pin, or tie that is passed through both openings thus coupling the both couplers 202a and 202b together.

In some embodiments of the clamp 200, couplers 202a and 202b are configured to couple in a buttoning fashion. In some embodiments, one of the couplers 202a and 202b comprises a button shape and the other coupler (either 202a or 202b) comprises an opening that is configured to securely fit over the button thus coupling both couplers 202a and 202b.

In some embodiments of the clamp 200, the clamp 200 includes one or more fixing elements 106 configured to securely fix the clamp 200 to the cervix of the patient. In some embodiments of clamp 200, one or more fixing elements 206 comprise one or more teeth as shown in FIG. 2. Embodiments of clamp 200 that include one or more fixing elements 206 are beneficial in that they, for example, secure the clamp 200 to the cervix of the patient in a manner that requires less pressure be applied by the first and second arms 208a, 208b against the walls of the cervix of the patient then would be applied if there were no fixing elements 206 and the first and second arms 208a, 208b alone secured the clamp 200 to the cervix of the patient. Having less pressure applied by the first and second arms 208a, 208b is beneficial in patients at risk for ischemia. Embodiments of clamp 200 that do not include any fixing elements 206 are beneficial in that they have a lower risk of causing bleeding from the cervix of the patient than the embodiments of clamp 100 that include the penetrating fixing elements 206, because these embodiments are configured to not penetrate the cervix of the patient.

In some embodiments of clamp 200, clamp 200 includes one or more sensors 210. The one or more sensors 210 are positioned so that they are able to sense physiologic parameters when the clamp 200 is positioned to encircle the cervix of the patient. In some embodiments of clamp 200, clamp 200 comprises a pressure sensor such as, for example, a tactile pressure sensor that is configured to sense a radial pressure exerted by the cervix of the patient against the sensor. A radial pressure exerted by the cervix of the patient that is sensed by the pressure sensor may, for example, indicate a contraction of the uterus or may, for example, indicate continued opening of the cervix of the patient. In some embodiments of clamp 200, clamp 200 includes a sensor configured to sense blood flow to the cervix of the patient such as, for example, a pulse oxygen sensor. A change in blood flow to the cervix may, for example, indicate that the clamp 200 is too tight and is constricting blood flow. In some embodiments of claim 200, clamp 200 comprises a fetal heart rate sensor configured to sense a heart rate of a fetus and thus provides information regarding the health of the fetus. It should be understood numerous other sensor types are suitable for use with the systems, devices, and methods described herein including and not limited to blood pressure sensors, glucose sensors, and heart rate sensors (sensing maternal heart rate). It should be also understood that some embodiments of clamp 200 include one sensor and some embodiments of clamp 200 include a plurality of sensors. In embodiments that include one sensor, the one sensor may sense either a single or multiple parameters, and in embodiments that include a plurality of sensors each of the sensors may sense either a single or multiple parameters. One or more sensors may positioned to be in contact with the cervix of the patient when the clamp 200 is positioned to encircle the cervix of the patient or the one or more sensors may be positioned in a different location within the pelvis (i.e. not in contact with the cervix of the patient) when the clamp 200 is positioned to encircle the cervix of the patient.

In some embodiments, a clamp 200 includes a transmitter that is functionally coupled with one or more sensors of a clamp 200. The transmitter is configured to transmit a wireless signal to a receiver that is positioned outside of the body of the patient.

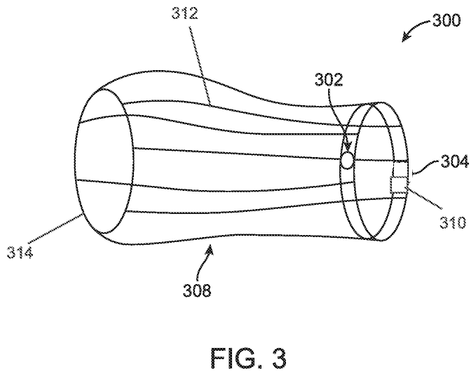

FIG. 3 shows an exemplary embodiment of a device 300 for closing a prematurely open cervix during pregnancy that includes a skirt 308. In some embodiments of device 300, the embodiments shown in either FIG. 1 or 2 and respectively described with reference to either FIG. 1 or 2 also comprise a skirt 308. As such, in some embodiments, device 300 includes any of the features described with reference to FIG. 1 or 2.

As shown in FIG. 3, a skirt 308 is coupled to clamp 304 and extends from clamp 304 to form an elongate cylinder shape. In these embodiments, when the clamp 304 is positioned to encircle the cervix of the patient, the skirt 308 also encircles the cervix of the patient. That is, the skirt 308 is coupled to the clamp 304 so that when the coupling mechanism 302 of clamp 304 is decoupled the skirt 308 is open as well such that the clamp 304 along with skirt 308 may be positioned to encircle at least a portion of the cervix of the patient.

In some embodiments, skirt 308 comprises elongate-struts 312 that extend from the clamp 304 and are each connected to one another at the opposite end of the device 300 by at least one cross-strut 314 that runs across and connects all of the elongate-struts 312. In some embodiments, the space between each of the elongate-struts 312 is covered by a membrane or material, and in some embodiments, the space between elongate-struts 312 is open.

In yet another alternative embodiment (not shown), device 300 does not include a coupler 302 so that the device 300 cannot be opened and is thus fixed in a cylindrical shape. In this embodiment, clamp 304 comprises either an elastic or malleable material so that it may be slid over a cervix and then either, in the case of the elastic material, the clamp 304 elastically fits around the cervix of the patient, or, in the case of the malleable material, is cinched around the cervix of the patient by a pressure applied by the healthcare provider. The skirt 308, in these coupler-less embodiments, may likewise be made of either an elastic or malleable material and may thus likewise be either elastically fitted or cinched around the cervix of the patient.

In some embodiments of device 300, device 300 includes one or more sensors 310. The one or more sensors 310 are positioned so that they are able to sense physiologic parameters when the device 300 is positioned to encircle the cervix of the patient. In some embodiments of device 300, device 300 comprises a pressure sensor such as, for example, a tactile pressure sensor that is configured to sense a radial pressure exerted by the cervix of the patient against the sensor. A radial pressure exerted by the cervix of the patient that is sensed by the pressure sensor may, for example, indicate a contraction of the uterus or may, for example, indicate continued opening of the cervix of the patient. In some embodiments of device 300, device 300 includes a sensor configured to sense blood flow to the cervix of the patient such as, for example, a pulse oxygen sensor. A change in blood flow to the cervix may, for example, indicate that the device 300 is too tight and is constricting blood flow. In some embodiments of device 300, device 300 comprises a fetal heart rate sensor configured to sense a heart rate of a fetus and thus provides information regarding the health of the fetus. It should be understood numerous other sensor types are suitable for use with the systems, devices, and methods described herein including and not limited to blood pressure sensors, glucose sensors, and heart rate sensors (sensing maternal heart rate). It should be also understood that some embodiments of device 300 include one sensor and some embodiments of device 300 include a plurality of sensors. In embodiments that include one sensor, the one sensor may sense either a single or multiple parameters, and in embodiments that include a plurality of sensors each of the sensors may sense either a single or multiple parameters. One or more sensors may positioned to be in contact with the cervix of the patient when the device 300 is positioned to encircle the cervix of the patient or the one or more sensors may be positioned in a different location within the pelvis (i.e. not in contact with the cervix of the patient) when the device 300 is positioned to encircle the cervix of the patient.

In some embodiments, a device 300 includes a transmitter that is functionally coupled with one or more sensors of a device 300. The transmitter is configured to transmit a wireless signal to a receiver that is positioned outside of the body of the patient.

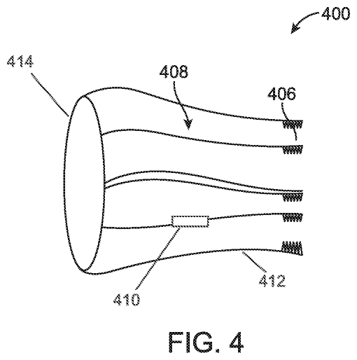

FIG. 4 shows an exemplary embodiment of a device 400 for closing a prematurely open cervix during pregnancy that includes a skirt 408 and does not include a clamp.

As shown in FIG. 4, skirt 408 comprises one or more elongate-struts 412 that are connected at each other via a cross-strut 414 that runs across and connects all of the elongate-struts 412. In some embodiments, the space between each of the elongate-struts 412 is covered by a membrane or material, and in some embodiments, the space between elongate-struts 412 is open.

At a distal end of device 400 are one or more fixing elements 406 positioned along one or more elongate-struts 412. In some embodiments, one or more fixing elements 406 comprise a series of penetrating teeth as shown in FIG. 4. In some embodiments, one or more fixing elements 406 each comprise a single penetrating tooth. In some embodiments, one or more fixing elements 406 are positioned at an end of one or more elongate struts 412 as shown in FIG. 4. In some embodiments, one or more fixing elements 406 are positioned proximally along the length of one or more elongate-struts 412 towards the proximal end of the device 400.

In some embodiments of the device 400, the one or more elongate-struts are configured to splay outwards and away from a horizontal longitudinal axis of the device 400.

In some embodiments of device 300, device 300 includes one or more sensors 410. The one or more sensors 410 are positioned so that they are able to sense physiologic parameters when the device 400 is positioned to encircle the cervix of the patient. In some embodiments of device 300, device 400 comprises a pressure sensor such as, for example, a tactile pressure sensor that is configured to sense a radial pressure exerted by the cervix of the patient against the sensor. A radial pressure exerted by the cervix of the patient that is sensed by the pressure sensor may, for example, indicate a contraction of the uterus or may, for example, indicate continued opening of the cervix of the patient. In some embodiments of device 400, device 400 includes a sensor configured to sense blood flow to the cervix of the patient such as, for example, a pulse oxygen sensor. A change in blood flow to the cervix may, for example, indicate that the device 300 is too tight and is constricting blood flow. In some embodiments of device 400, device 400 comprises a fetal heart rate sensor configured to sense a heart rate of a fetus and thus provides information regarding the health of the fetus. It should be understood numerous other sensor types are suitable for use with the systems, devices, and methods described herein including and not limited to blood pressure sensors, glucose sensors, and heart rate sensors (sensing maternal heart rate). It should be also understood that some embodiments of device 400 include one sensor and some embodiments of device 300 include a plurality of sensors. In embodiments that include one sensor, the one sensor may sense either a single or multiple parameters, and in embodiments that include a plurality of sensors each of the sensors may sense either a single or multiple parameters. One or more sensors may positioned to be in contact with the cervix of the patient when the device 300 is positioned to encircle the cervix of the patient or the one or more sensors may be positioned in a different location within the pelvis (i.e. not in contact with the cervix of the patient) when the device 400 is positioned to encircle the cervix of the patient.

In some embodiments, a device 400 includes a transmitter that is functionally coupled with one or more sensors of a device 400. The transmitter is configured to transmit a wireless signal to a receiver that is positioned outside of the body of the patient.

Systems for Treating and Monitoring a Pregnant Patient with a Prematurely Open Cervix

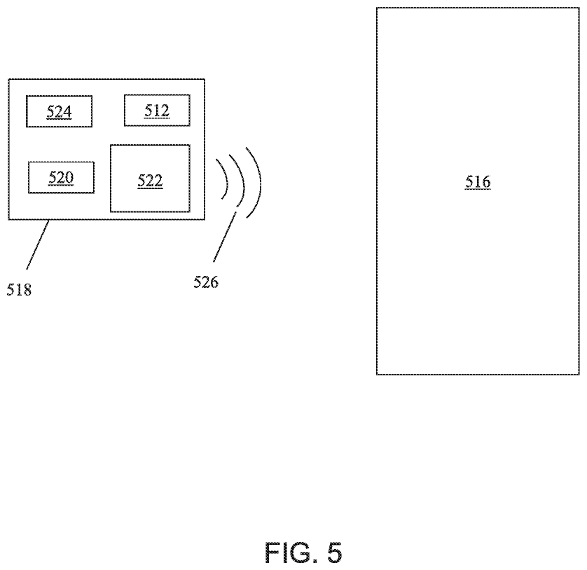

FIG. 5 shows an exemplary embodiment of a system for treating and monitoring a pregnant patient with a prematurely open cervix during pregnancy. As described herein, one or more sensors 510 may be incorporated into any of the embodiments of devices described herein for treating and monitoring a pregnant patient having a prematurely open cervix. In some embodiments of the devices described herein, one or more sensors 510 is a component of a sensor assembly 518 that is coupled with or integrated with any of the embodiments of devices for treating and monitoring a pregnant patient having a prematurely open cervix described herein.

In some embodiments, a sensor assembly 518 includes one or more sensors 510, a power source 520, and a transmitter 522 configured to transmit a wireless signal 526. In some embodiments of the sensor assembly 518, the sensor assembly 518 also includes a processor 524. A sensor assembly 510 is configured to sense a physiologic parameter of a patient, and transmit the physiologic parameter in the form of an electronic data signal to an electronic device 516 located outside of the body of the patient. The electronic device 516 may be a mobile electronic device such as a smartphone, tablet computer, or smartwatch. The components of a sensor assembly 518 may be integrated into a single unit that is coupled to or integrated with a device for treating and monitoring a pregnant patient having a prematurely open cervix as described herein.

In some embodiments, a single sensor assembly 518 includes a single sensor 510 and in some embodiments, a single sensor assembly 518 includes two or more sensors 510. In some embodiments of the devices described herein for treating and monitoring a pregnant patient having a prematurely open cervix, the device includes more than one sensor assembly 518.

A power source 520 comprises a battery in some embodiments of the sensor assembly 518, and it is configured to power any electronic components of the sensor assembly 518 requiring a power source.

Transmitter 522 is configured to wirelessly transmit an electronic data signal to a receiver located outside of the body of the patient. Numerous types of signals are suitable for transmission via different embodiments of the transmitter 522, non-limiting examples of which include Wi-Fi, ultrasound signals, Bluetooth signals, acoustic signals, and radiofrequency signals.

In some embodiments, a sensor assembly 518 further includes a processor 524. Processor 524 is operatively coupled with the one or more sensors 510 and may be configured to process physiologic parameter data received from the one or more sensors 510. In some embodiments, processor 524 converts a sensed physiologic data signal received from a sensor into a transmittable signal that is then transmitted to transmitter 522 for transmission to one or more electronic devices 516.

In some embodiments of the systems described herein, an electronic device 516 is configured communicate with one or more remote computer systems or a host through the network 526. In some embodiments, an electronic device 516 may be, for example, an electronic device 516 of a healthcare provider used to monitor a patient having a device as described herein placed to encircle their cervix. The electronic device 516 includes a receiver for receiving a transmitted signal transmitted from either transmitter 522 or from a remote host or other electronic device via network 526. In some embodiments of the system described herein, an electronic device 516 comprises a host such as, for example, a computing device located at a remote monitoring facility that is configured to monitor one or more patients having a device as describe herein placed to encircle their cervix.

In some embodiments of the system described herein, an electronic device 516 includes software that is configured to cause a processor of the electronic device 516 to analyze and/or display data associated with the condition of the patient having the device as described herein placed to encircle their cervix.

In some embodiments of the electronic device 516, software is configured to analyze sensed data by comparing a sensed data value sensed by sensor 510 to a normal value, a threshold value, and/or a range of values, and is further configured to alert a user of the electronic device 516 if the sensed data value differs from normal, is beyond (or below) the threshold value, and/or is outside of a normal range of values.

In some embodiments of the electronic device 516, a sensed data value sensed by sensor 510 is analyzed to determine a condition of a patient or a fetus. For example, if a sensed value of blood flow is determined by the software to be abnormal, the software determines that the device enclosing the cervix is to too tight and is causing ischemia.

In some embodiments, more than one type of sensed data value is used to determine a condition of a patient and determine a treatment. For example, a sensed decrease in blood flow to the cervix is considered along with an increase in a radial pressure exerted by the cervix against the device encircling the cervix. In response to the sensed decrease in blood flow and increase in exerted pressure, the software determines that the patient's cervix is dilating against a device that is too tight. The software determines that patient should be examined, and alerts the healthcare provider to do so along with suggesting possible removal or adjustment of the device to the healthcare provider before ischemia is caused. Similarly, for example, a sensed normal amount of blood flow to the cervix may is considered along with a sensed increase in a radial pressure exerted by the cervix against the device encircling the cervix. The sensed normal blood flow to the cervix together with the sensed increased radial pressure exerted by the cervix causes the software to determine that the patient's cervix continues to dilate and the device encircling the cervix is likely not applying enough resistance to prevent the further dilation. The software determines that patient should be examined, and alerts the healthcare provider to do so along with suggesting possible tightening or repositioning of the device encircling the cervix.

Methods for Treating and Monitoring a Pregnant Patient with a Prematurely Open Cervix

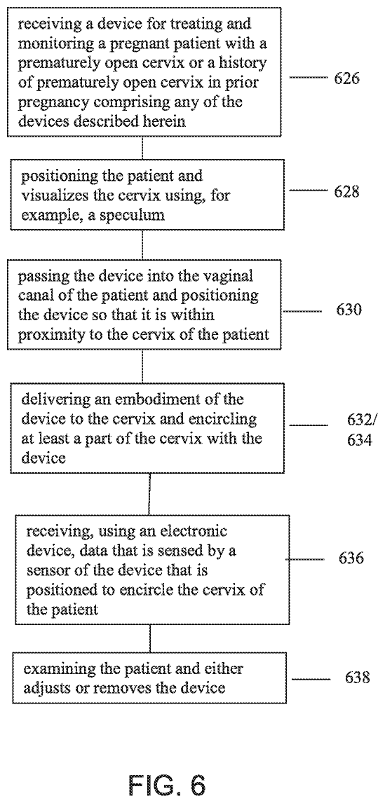

FIG. 6 shows the steps of an exemplary method for treating and monitoring a pregnant patient with a prematurely open cervix. In a step 626, a healthcare provider receives a device for treating and monitoring a pregnant patient with a prematurely open cervix or a history of prematurely open cervix in prior pregnancy comprising any of the devices described herein (“the device” hereafter). The device may be provided to the health care provider as part of a kit that further includes other instruments to aid in the placement of the device such as, for example, a delivery instrument configure to hold the device and place it so that it encloses the cervix. In a step 628, a healthcare provider positions the patient and visualizes the cervix using, for example, a speculum. In a step 630, the healthcare provider passes the device into the vaginal canal of the patient and positions the device so that it is within proximity to the cervix of the patient. In a step 632, a healthcare provider delivers an embodiment of the device to the cervix that is configured to open and close (e.g. an embodiment with a coupling mechanism), the healthcare provider encircles the cervix with the open device, and then the healthcare provider closes the device so that the closed device encircles at least a part of the cervix and is secured to the cervix either through one or more fixing elements and/or through pressure applied by the closed device against the walls of the cervix. In an alternative to step 632, a healthcare provider in a step 634, delivers an embodiment of the device to the cervix that is not configured to open and close, and the healthcare provider slides the device up the cervix and secures the device to the cervix either through one or more fixing elements and/or through pressure applied by the closed device against the walls of the cervix.

When the healthcare provider is using an embodiment of a system as described herein, in a step 636 the healthcare provider receives, using an electronic device, data that is sensed by a sensor of the device that is positioned to encircle the cervix of the patient. The data received by the electronic device may be received directly from a transmitter coupled to the sensor or the transmitter coupled with sensor may transmit sensed data to a remote host computer that then transmits data (either sensed data or analyzed data) to the electronic device of the healthcare provider. If the data received by the electronic device indicates (e.g. through an alarm or alert) that a sensed value or determined condition is abnormal, in a step 638 the healthcare provider examines the patient and either adjusts or removes the device. An adjustment may comprise, for example, re-coupling the coupling elements of the device so that the device does not apply the same pressure to the cervix.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device for preventing a pre-mature opening of a cervix of a patient, comprising:
a first arm having a first proximal end and a first distal end;
a second arm having a second proximal end and a second distal end;
a hinge that rotatably couples the first arm at the first proximal end with the second arm at the second proximal end, the first arm and the second arm being positioned so that the first arm and the second arm surround the cervix of the patient when the first arm and the second arm are rotated towards each other about the hinge;
a first coupler positioned at the first distal end and a second coupler positioned at the second distal end, wherein the first coupler and the second coupler are configured to couple with each other such that the first arm and the second arm are secured against the cervix of the patient; and
a sensor configured to sense a change associated with the cervix of the patient.

2. The device of claim 1, wherein the first arm and the second arm each have one or more teeth for securing the first arm and the second arm to the cervix of the patient.

3. The device of claim 1, wherein the first arm and the second arm each comprises a curved segment configured to conform to the shape of the cervix of the patient.

4. The device of claim 1, wherein the first coupler and the second coupler are configured to reversibly couple with each other.

5. The device of claim 1, wherein the sensor comprises a pressure sensor configured to sense a pressure exerted by the cervix of the patient on the device.

6. The device of claim 5, wherein the device comprises a transmitter configured to transmit the pressure exerted by the cervix of the patient on the device that is sensed by the sensor.

7. The device of claim 1, wherein the first coupler comprises an opening having one or more first protrusions and first recesses and the second coupler comprises a flat segment configured to fit the opening and having one or more second protrusions and second recesses, and wherein the first coupler and second coupler couple in a ratcheting fashion wherein the first protrusions couple with the second recesses and the first recesses couple with the second protrusions.

8. A method for preventing a pre-mature opening of a cervix of a patient comprising:
securing a clamp, having a sensor, around the cervix of the patient without use of a suture, thereby closing an opening of the cervix of the patient; and
monitoring, using the sensor, a change associated with the cervix of the patient.

9. The method of claim 8, wherein the clamp comprises a first arm having a first proximal end and a first distal end, a second arm having a second proximal end and a second distal end, a hinge that rotatably couples the first arm at the first proximal end with the second arm at the second proximal end, the first arm and the second arm being positioned so that the first arm and the second arm surround the cervix of the patient when the first arm and the second arm are rotated towards each other about the hinge, a first coupler positioned at the first distal end, and a second coupler positioned at the second distal end, wherein the first coupler and the second coupler are configured to couple with each other such that the first arm and the second arm are secured against the cervix of the patient.

10. The method of claim 9, wherein the first arm and the second arm each have one or more teeth for securing the first arm and the second arm to the cervix of the patient.

11. The method of claim 9, wherein the first arm and the second arm each comprises a curved segment configured to conform to the shape of the cervix of the patient.

12. The method of claim 9, wherein the first coupler and the second coupler are configured to reversibly couple with each other.

13. The method of claim 8, wherein the sensor comprises a pressure sensor configured to sense a pressure exerted by the cervix of the patient on the clamp.

14. The method of claim 8, wherein the clamp comprises a transmitter configured to transmit the pressure exerted by the cervix of the patient on the device that is sensed by the sensor.

15. The method of claim 8, wherein the first coupler comprises an opening having one or more first protrusions and first recesses and the second coupler comprising a flat segment configured to fit the opening and having one or more second protrusions and second recesses, and wherein the first coupler and second coupler couple in a ratcheting fashion wherein the first protrusions couple with the second recesses and the first recesses couple with the second protrusions.

16. A system for preventing a pre-mature opening of a cervix of a patient comprising:
a clamp comprising:
a first arm having a first proximal end and a first distal end;
a second arm having a second proximal end and a second distal end;
a hinge that rotatably couples the first arm at the first proximal end with the second arm at the second proximal end, the first arm and the second arm being positioned so that the first arm and the second arm surround the cervix of the patient when the first arm and the second arm are rotated towards each other about the hinge;
a first coupler positioned at the first distal end and a second coupler positioned at the second distal end, wherein the first coupler and the second coupler are configured to couple with each other such that the first arm and the second arm are secured against the cervix of the patient;
a sensor configured to sense data associated with the cervix of the patient.
a transmitter configured to receive the data from the sensor and wirelessly transmit the data;
a host comprising:
a receiver configured to receive the data that is transmitted by the transmitter;
a processor; and
a non-transitory computer-readable storage media encoded with a computer program including instructions executable by the processor to cause the processor:
determine if the data is within pre-set limits; and
generate an alert when the data is outside of the pre-set limits.

17. The system of claim 16, wherein the first arm and the second arm each have one or more teeth for securing the first arm and the second arm to the cervix of the patient.

18. The system of claim 16, wherein the first arm and the second arm each comprises a curved segment configured to conform to the shape of the cervix of the patient.

19. The system of claim 16, wherein the first coupler and the second coupler are configured to reversibly couple with each other.

20. The system of claim 16, wherein the sensor comprises a pressure sensor configured to sense a pressure exerted by the cervix of the patient on the device.

21. The system of claim 20, wherein the device comprises a transmitter configured to transmit the pressure exerted by the cervix of the patient on the device that is sensed by the sensor.

22. The system of claim 1, wherein the first coupler comprises an opening having one or more first protrusions and first recesses and the second coupler comprises a flat segment configured to fit the opening and having one or more second protrusions and second recesses, and wherein the first coupler and second coupler couple in a ratcheting fashion wherein the first protrusions couple with the second recesses and the first recesses couple with the second protrusions.

23. The system of claim 1, wherein the host comprises a mobile computing device

24. The system of claim 23, wherein the mobile computing device comprises a smartwatch or smartphone.
